(19) United States
(12) Patent Application Publication
(10) Pub. No.: US 2024/0136021 A1
(43) Pub. Date: Apr. 25, 2024

(54) PERCEPTUAL REPRESENTATION LEARNING METHOD FOR PROTEIN CONFORMATIONS BASED ON PRE-TRAINED LANGUAGE MODEL

(71) Applicant: ZJU-HANGZHOU GLOBAL SCIENTIFIC AND TECHNOLOGICAL INNOVATION CENTER, HANGZHOU, Zhejiang Province (CN)

(72) Inventors: QIANG ZHANG, HANGZHOU, ZHEJIANG PROVINCE (CN); ZEYUAN WANG, HANGZHOU, ZHEJIANG PROVINCE (CN); YUQIANG HAN, HANGZHOU, ZHEJIANG PROVINCE (CN); HUAJUN CHEN, HANGZHOU, ZHEJIANG PROVINCE (CN)

(21) Appl. No.: 18/278,298

(22) PCT Filed: Oct. 20, 2022
(86) PCT No.: PCT/CN2022/126696

§ 371 Date: Aug. 21, 2023

(30) Foreign Application Priority Data
Feb. 9, 2022 (CN) 202210122014.5

(51) Int. Cl. G16B 40/00; G16B 5/00

(52) U.S. Cl. CPC G16B 40/00; G16B 5/00

(57) ABSTRACT

The present invention discloses a perceptual representation learning method for protein conformations based on a pre-trained language model, including: obtaining a protein made up of an amino acid sequence, building different data sets according to protein conformations, and defining a prompt for each type of protein conformation; building, based on a pre-trained language model, a representation learning module for fusing an embedding representation of each type of the prompt into an embedding representation of the protein, so as to obtain a protein embedding representation under a prompt identifier; building a task module for performing task prediction on a task corresponding to each type of protein conformation based on the protein embedding representation under the prompt identifier; building a loss function for each type of task based on a task prediction result and a tag, and updating model parameters of the representation learning module and the task module in combination with loss functions of all types of tasks and the different data sets; and after the model parameters are updated, extracting the representation learning module as a protein representation module. Protein representations under different conformations can be obtained by the method.

### TECHNICAL FIELD

The present invention belongs to the technical field of representation learning, and specifically relates to a perceptual representation learning method for protein conformations based on a pre-trained language model.

### BACKGROUND TECHNOLOGY

A protein is sequence data consisting of several amino acids. At present, in many researches, embedding representations of proteins are learnt by using language models in natural languages to predict their structures and properties.

According to an unsupervised learning method, initial parameters are provided for a model by using a masked language model object and are finely adjusted on a tagged data set together with a mapping function, and a representation of a protein in a task space is learnt and applied to a downstream prediction task. As a class of macromolecule, the protein has different conformations in different environments, for example, it may be spontaneously folded through chemical properties of amino acids or may make morphological changes through contact with other proteins. An existing protein language model can only provide a unique embedding matrix for a specific protein, making it difficult to handle the effects in both a prediction task for a structure and a prediction task for a function, which limits the expressive ability of the model and the application of the model in practical scenarios.

The successful representation of prompt ideas in natural languages indicates that language models have good expressive abilities and can have a desired impact on overall output by changing local symbols of a sequence. Conventional prompt ideas are to find optimal prompts in a semantic space by using existing pre-trained models. In a protein pre-training model, semantic units are amino acids, which cannot be manually built like language prompts. In addition, prompts on a continuous space lack interpretability.

The patent document CN106845149A discloses a new method for representing a protein sequence based on gene ontology information. First, a Swiss-Prot database is searched using a BLAST program to find all similar protein sequences of a protein sequence P, all proteins in a training data set are input to a GO database, and GO ontology information of each protein is searched; then, a gene ontology database is searched for tagged gene ontology information of the protein P; and the protein P is defined as a discrete vector of M elements according to M tags of a prediction problem, that is, the sequence representation dimension of the protein is reduced by fusing the GO information of the protein in a sequence set is fused into a new vector description of the protein P.

The patent document CN104134017A discloses a method for extracting a protein interaction pair based on a compact feature representation, including the following steps: 1) selecting a required corpus, which is based on sentences and already has annotations of protein entities and annotations of entity relationships; 2)discarding sentences in step 1) that do not contain the protein entities or only contain one protein entity, to obtain a set of sentences; 3) replacing the corresponding protein entities in the sentences with placeholders and performing placeholder fusion, followed by part-of-speech tagging and syntactic analysis; 4) obtaining features of words, parts of speech, syntax, and templates based on each entity pair; 5) performing operation of compact expression on the features obtained in step 4); and 6) training the features obtained in step 4) by using a support vector machine or performing prediction using a trained model. The method is to increase the amount of information in feature vectors by making the best use of available information in sentences.

Although the above two patent applications have their respective advantages, there is still a problem of inaccurate representation vectors obtained due to no consideration of protein conformations, which limits the effects in prediction tasks for protein structure and function.

### SUMMARY OF THE INVENTION

In view of the above, an object of the present invention is to provide a perceptual representation learning method for protein conformations based on a pre-trained language model. By a combined pre-training method for a plurality of data sets, protein representations under different conformations are obtained, thereby improving the accuracy of prediction tasks for protein structure and function.

To achieve the above object of the invention, an embodiment of the present invention provides a perceptual representation learning method for protein conformations based on a pre-trained language model, including the following steps:
obtaining a protein made up of an amino acid sequence, building different data sets according to protein conformations, and defining a prompt for each type of protein conformation;
building, based on a pre-trained language model, a representation learning module for fusing an embedding representation of each type of prompt into an embedding representation of the protein, so as to obtain a protein embedding representation under a prompt identifier;
building a task module for performing task prediction on a task corresponding to each type of protein conformation based on the protein embedding representation under the prompt identifier;
building a loss function for each type of task based on a task prediction result and a tag, and updating model parameters of the representation learning module and the task module in combination with loss functions of all types of tasks and the different data sets; and
after the model parameters are updated, extracting the representation learning module as a protein representation module.

In an embodiment, the representation learning module includes a prompt embedding layer, an amino acid embedding layer, a fusion layer, and the pre-trained language model, wherein the prompt embedding layer is configured to learn the embedding representation of each type of prompt, the amino acid embedding layer is configured to learn the embedding representation of the protein, the fusion layer is configured to fuse the embedding representation of each type of prompt and the embedding representation of protein to obtain a fused representation, and the pre-trained language model is configured to perform representation learning on the fused representation to obtain the protein embedding representation under each type of prompt identifier; and
the task module includes a task mapping layer corresponding to each type of protein conformation, and each task mapping layer is configured to perform task prediction based on the protein embedding representation under each type of prompt identifier.

In an embodiment, the amino acid embedding layer includes an amino acid information embedding layer and an amino acid position embedding layer which are configured to extract an amino acid information representation and an amino acid position representation according to the amino acid sequence, respectively, and the amino acid information representation and the amino acid position representation are superimposed to obtain the embedding representation of the protein.

In an embodiment, the pre-trained language model is a pluggable masked pre-trained language model, where the masked pre-trained language model comprises BERT, RoBERTa, ALBERT, and XLNet.

In an embodiment, the task mapping layer includes a dual-layer MLP for performing task prediction based on the protein embedding representation under each type of prompt identifier.

In an embodiment, the loss function for each type of prediction task is to minimize an error of the task prediction result and the tag.

In an embodiment, the protein representation module is applied to a prediction task for a protein structure and/or a protein function; during application, in the protein representation module, embedding representations of all types of prompts are simultaneously fused into the embedding representation of the protein, so as to obtain protein embedding representations under all prompt identifiers; and the protein embedding representations under all the prompt identifiers are configured to predict the protein structure and/or the protein function.

In an embodiment, the embedding representations of all types of prompts being simultaneously fused into the embedding representation of the protein includes:
splicing the embedding representations of all the types of prompts first, and then performing fusion on an obtained spliced representation and the embedding representation of the protein, wherein the fusion includes splicing, full connection mapping, and convolutional mapping.

In an embodiment, the protein conformation includes a natural folding state and an interaction state;
for the natural folding state, an amino acid sequence with a mask is taken as a sample and a normal amino acid sequence is taken as a sample tag to constitute a data set, a corresponding prompt in the natural folding state is a sequence prompt, and a corresponding task is a prediction task for a masked amino acid; and
for the interaction state, at least two amino acid sequences are taken as samples and a protein reaction type is taken as a sample tag to constitute a data set, a corresponding prompt in the interaction state is an interaction prompt, and a corresponding task is a prediction task for a protein interaction.

Compared with the prior art, the present invention has at least the following beneficial effects:

The different data sets centered on the protein conformations are built based on protein data, and the prompts are added to reflect the protein conformations and to learn the protein embedding representations under the prompt identifiers. On this basis, different prediction tasks are performed using the protein embedding representations under different prompt identifiers to cause the pre-trained language model to calculate a sum of losses from the plurality of different prediction tasks, so as to learn more discriminative conformation prompt representations. The protein embedding representations fused with the conformation prompt representations can improve the accuracy of prediction tasks for protein structure and function.

### BRIEF DESCRIPTION OF THE DRAWINGS

To more clearly illustrate the technical solutions in the embodiments of the present invention or in the prior art, the accompanying drawings that need to be used in the description of the embodiments or the prior art will be briefly described below. Apparently, the accompanying drawings in the description below merely illustrate some embodiments of the present invention. Those of ordinary skill in the art may also derive other accompanying drawings from these accompanying drawings without creative efforts.

FIG. 1 is a flowchart of a perceptual representation learning method for protein conformations based on a pre-trained language model;

FIG. 2 is a schematic diagram of a process for building a protein representation module provided by an embodiment; and

FIG. 3 is a schematic diagram of a process for applying the protein representation module provided by the embodiment.

### DETAILED DESCRIPTION OF THE EMBODIMENTS

To achieve the object, the technical solutions and advantages of the present invention clearer, the present invention is further described in detail below in conjunction with the accompanying drawings and the embodiments. It should be understood that the specific embodiments described herein are merely used to explain the present invention, but not to limit the scope of protection of the present invention.

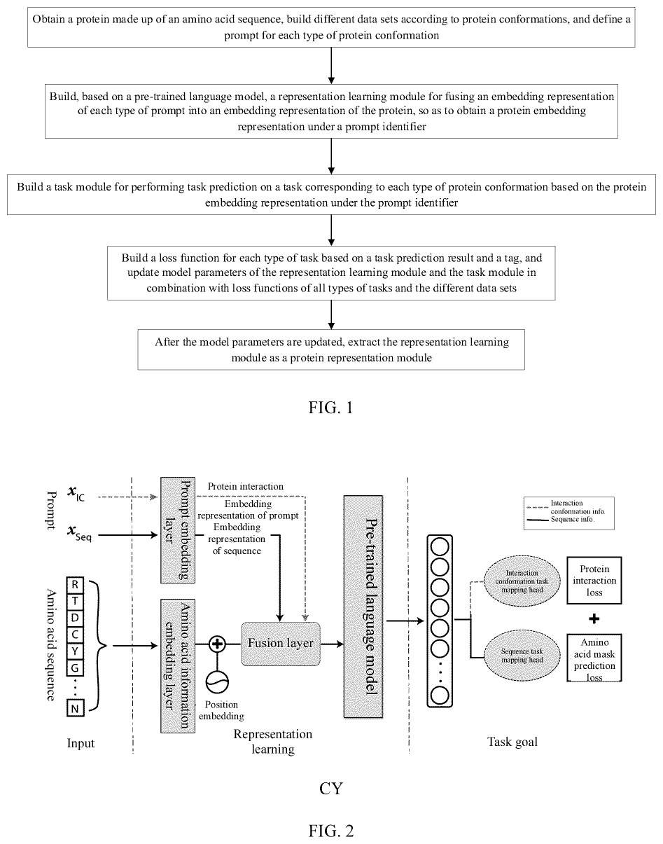

FIG. 1 is a flowchart of a perceptual representation learning method for protein conformations based on a pre-trained language model. As shown in FIG. 1, the perceptual representation learning method for protein conformations based on a pre-trained language model provided by an embodiment includes steps below.

In step 1, a protein made up of an amino acid sequence is obtained, different data sets are built according to protein conformations, and a prompt is defined for each type of protein conformation.

In this embodiment, protein structure data is obtained based on an experimental result for the protein in the biological field, where each piece of the protein structure data includes the amino acid sequence; and protein function data, namely, data of an interaction between proteins is obtained while the protein structure data is obtained, where the interaction data is a protein reaction type.

The protein conformations refer to different states of the protein and include a natural folding and an interaction state. After the protein data is obtained, the data sets showing different properties are built based on different protein conformations to provide a basis for protein data processing centered on the protein conformations, so as to perform protein embedding representation learning on different prediction tasks.

In this embodiment, for the natural folding state, an amino acid sequence with a mask is taken as a sample and a normal amino acid sequence (an original amino acid sequence without a mask) is taken as a sample tag to constitute an amino acid sequence data set. A prediction task for a masked amino acid is built based on the amino acid sequence data set, that is, the prediction task for the masked amino acid is performed using the amino acid sequence data set, so as to learn an embedding representation of the protein in the natural folding state.

In this embodiment, for the interaction state, at least two interacting amino acid sequences are taken as samples and the protein reaction type is taken as a sample tag to constitute a protein interaction data set. A prediction task for a protein interaction is built based on the protein interaction data set, that is, the prediction task for the protein interaction is performed using the protein interaction data set, so as to learn an embedding representation of the protein in the interaction state.

In this embodiment, interacting proteins in the protein interaction data set are represented in the form of a network graph; after the network graph is obtained, the network graph is cut according to species, and proteins that cannot be used as inputs are removed according to the pre-trained language model; and for each step in training, a protein interaction network of a species is selected, a protein with a total length of no more than 2,048 amino acids is randomly sampled, and an interaction matrix of subgraphs is obtained for input of a representation learning model.

The protein reaction type includes reaction, expression, activation, post-translational modification, binding, and catalysis. When the protein interaction data set is built, at least a protein reaction type is used as a tag.

In this embodiment, to mark the protein conformations, the prompt is defined for each type of protein conformation, that is, a prompt x_{Seq} is defined for the natural folding state, and a prompt x_{IC} is defined for the interaction state. The prompt is fused into the embedding representation of the protein during learning, so as to mark the protein conformation.

In step 2, a representation learning module is, based on a pre-trained language model, built for fusing an embedding representation of each type of prompt into an embedding representation of the protein, so as to obtain a protein embedding representation under a prompt identifier.

In this embodiment, during building of the protein embedding representation, the prompt representing the protein conformation is added into a vocabulary of the pre-trained language model; and when the protein embedding representation is input to the pre-trained language model and carries protein conformation information, the pre-trained language model will add the embedding representation of the conformation prompt into the protein embedding representation.

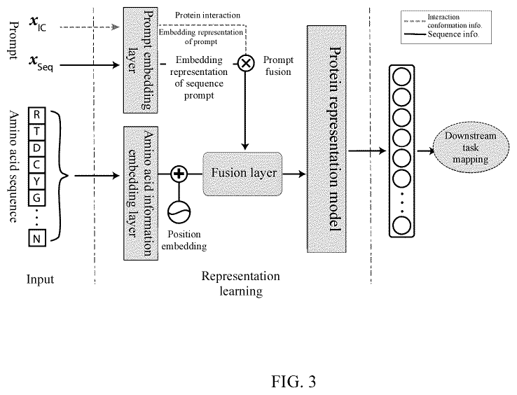

In an implementation, as shown in FIG. 2, the representation learning module includes a prompt embedding layer, an amino acid embedding layer, a fusion layer, and the pre-trained language model, wherein the prompt embedding layer is configured to map each type of prompt to a high-dimensional space, so as to learn the embedding representation of each type of prompt; the amino acid embedding layer is configured to map the protein to the high-dimensional space, so as to learn the embedding representation of the protein; the fusion layer is configured to fuse the embedding representation of each type of prompt and the embedding representation of protein to obtain a fused representation, and the fusion layer may perform fusion of the embedding representations by means of splicing, full connection mapping, and convolutional mapping; and the pre-trained language model is configured to perform representation learning on the fused representation to obtain the protein embedding representation under each type of prompt identifier.

In an embodiment, as shown in FIG. 2, the amino acid embedding layer includes an amino acid information embedding layer and an amino acid position embedding layer which are configured to extract an amino acid information representation and an amino acid position representation according to the amino acid sequence, respectively, and the amino acid information representation and the amino acid position representation are superimposed (symbol + in FIG. 2) to obtain the embedding representation of the protein. The amino acid information representation represents information of amino acids that make up the protein, and the amino acid position representation represents position information of each amino acid that makes up the protein.

In this embodiment, the pre-trained language model is a pluggable masked pre-trained language model, wherein the masked pre-trained language model includes BERT, RoBERTa, ALBERT, and XLNet. Preferably, the masked pre-trained language model may be RoBERTa. These pre-trained language models perform representation learning on the input fused representation to obtain the protein embedding representation under each type of prompt identifier.

In step 3, a task module is built for performing task prediction on a task corresponding to each type of protein conformation based on the protein embedding representation under the prompt identifier.

In this embodiment, the task module includes a task mapping layer corresponding to each type of protein conformation, and each task mapping layer maps protein embedding under each type of prompt identifier to different task spaces by using different mapping functions, that is, each task mapping layer performs different task predictions. Preferably, the task mapping layer may perform different task predictions by using a dual-layer MLP as a mapping function.

For the corresponding task in the natural folding state is the prediction task for the masked amino acid and the corresponding task in the interaction state is the prediction task for the protein interaction, the task module includes two task mapping layers, which are an interaction conformation task mapping head and a sequence task mapping head, respectively, where each task mapping layer uses the dual-layer MLP as the mapping function.

In step 4, a loss function for each type of task is built based on a task prediction result and a tag, and model parameters of the representation learning module and the task module are updated in combination with loss functions of all types of tasks and the different data sets.

In this embodiment, the loss function for each type of prediction task is to minimize an error of the task prediction result and the tag. Then, the model parameters of the representation learning module and the task module are optimized on the data set corresponding to each type of prediction task in combination with the loss functions of all the types of prediction tasks, that is, the parameters of the prompt embedding layer, the amino acid embedding layer, the fusion layer, the pre-training language model, and the task mapping layer are optimized.

In this embodiment, when the protein conformation includes the natural folding state and the interaction state, it indicates that during learning, a built total loss function includes two parts of a loss of the prediction task for the masked amino acid and a loss of the prediction task for the protein interaction. Specifically,
in the prediction task for the masked amino acid, a training batch includes N proteins, and for each amino acid chain, 15% of amino acids are randomly selected for prediction. For each amino acid used for prediction, there is an 80% probability for it to become a [Mask] symbol, a 10% probability for it to become another amino acid symbol, and a 10% probability for it to remain unchanged; and sample data is built in this way to obtain a protein pair (P, P′), where P′ is a modified protein, and P is an original protein and serves as a tag of the protein P′ in the prediction task for the masked amino acid. Based on this, a loss function lᵢ(x, y) for a prediction task for an i^{th} amino acid in a protein is as follows:

$${l}_{i} ( x , {y}_{i} ) = - log \frac{exp ( {x}_{{y}_{i}} )}{{\sum }_{c = 1}^{C} exp ( {x}_{c} )}$$

wherein x represents a prediction result, x_{y}_{ᵢ} represents a probability that the prediction result is a true tag yᵢ, x_{c} represents a probability that the prediction result is a type c, C represents the total number of predicted types, and the total loss L_{S} of the prediction task for the masked amino acid is a sum of losses of prediction for all amino acids in a training batch, that is, L_{S}=Σ_{y}_{ᵢ}_{^{∈Y}}−log lᵢ(x,yᵢ).

In the prediction task for the protein interaction, a training batch includes N proteins; an average value of all amino acid embeddings in the protein is used as the embedding representation of the protein to predict whether there is an interaction between any two proteins; a loss function for the task is similar to a loss function for the above prediction task for the masked amino acid; and X={x₁, x₂, . . . , x_{N}} is set as a sampled protein sequence, y ∈{0,1}^{n×n} is an interaction matrix, and a loss function L_{PPI} is expressed as follows:

$${L}_{PPI} = {\sum }_{{x}_{i} ∈ X} {\sum }_{{x}_{j} ∈ X} BCE ( p ( {y}_{ij} \vert {x}_{i} , {x}_{j} ) )$$

wherein i≠j, both i and j are protein indexes, BCE represents a binary cross entropy, and p(yᵢ_{ⱼ}|xᵢ, xⱼ) represents a probability of correctly predicting an interaction between a protein xᵢ and a protein xⱼ to be a tag yᵢ_{ⱼ}.

Then the total loss is L=L_{S}+λL_{PPI}, where A is a hyperparameter.

In this embodiment, the amino acid sequence data set corresponding to the prediction task for the masked amino acid is input to the representation learning module, the protein interaction data set corresponding to the prediction task for the protein interaction is input to the representation learning module, and the model parameters of the representation learning module and the task module are updated with a goal of minimizing the total loss function including the loss of the prediction task for the masked amino acid and the loss of the prediction task for the protein interaction.

In step 5, after the model parameters are updated, the representation learning module is extracted as a protein representation module.

In this embodiment, after the model parameters are updated, the prompt embedding layer, the amino acid embedding layer, the fusion layer, and the pre-trained language model included in the representation learning module with the determined parameters are extracted as the protein representation module, where the pre-trained language model with the determined parameters is used as a protein representation model.

In this embodiment, the obtained protein representation module is applied to a prediction task for a protein structure and/or a protein function. During application, in the protein representation module, embedding representations of all types of prompts are simultaneously fused into the embedding representation of the protein, so as to obtain protein embedding representations under all prompt identifiers; and the protein embedding representations under all the prompt identifiers are configured to predict the protein structure and/or the protein function.

In an implementation, embedding representations of all types of prompts being simultaneously fused into the embedding representation of the protein includes: splicing the embedding representations of all the types of prompts first, and then performing fusion on an obtained spliced representation and the embedding representation of the protein, where the fusion includes splicing, full connection mapping, and convolutional mapping.

In an implementation, as shown in FIG. 3, the protein representation module includes a prompt embedding layer, an amino acid embedding layer, a fusion layer, and the protein representation model. When the protein representation module is applied to the prediction task for the protein structure and/or the protein function, a classifier (a downstream task mapping head) related to the prediction task is added after the protein representation model, where the protein representation model and the classifier constitute an end-to-end task prediction model.

During application, appropriate prompts are selected according to the prediction task, and a small data set for the prediction task is built; a protein and prompts included in the small data set are used as inputs of the task prediction model, and embedding representations of all types of prompts extracted by the prompt embedding layer are spliced to form a spliced representation, and the spliced representation is input to the fusion layer; the fusion layer performs fusion on an embedding representation of the protein and the spliced representation, then a fused representation is input to the protein representation model, and representation learning is performed to obtain protein embedding representations under all prompt identifiers; parameters of the classifier included in the task prediction model are finely adjusted based on the protein embedding representations; and therefore the task prediction model subjected to the parameter fine adjustment can predict the protein function and the protein structure.

Specifically, for two types of protein conformations in the natural folding state and the interaction state, prompts x_{Seq} and x_{IC} corresponding to the two types of protein conformations are input to the prompt embedding layer, respectively, so as to extract corresponding embedding representations; the two embedding representations are spliced and then a spliced embedding representation is input to the fusion layer; the spliced embedding representation and the embedding representation of the protein are fused in the fusion layer and then a fused embedding representation is input to the protein representation model, so as to obtain protein embedding representations under two prompt identifiers.

According to the perceptual representation learning method for protein conformations based on a pre-trained language model provided by the above embodiment, a pre-trained data set centered on protein conformations is first built based on disclosed protein data.

According to the perceptual representation learning method for protein conformations based on a pre-trained language model provided by the above embodiment, unlike the existing pre-trained language model, the protein representation generated by the method is unique and cannot reflect related information of the protein in different environments. The proposed protein representation method not only can reflect conformation information of protein interactions and protein spontaneous folding by adding conformation symbols, but also supports incremental addition of new conformation information under specific conditions.

In the perceptual representation learning method for protein conformations based on a pre-trained language model provided by the above embodiment, the conformation embedding and fusion methods used provide undifferentiated conformation information for all amino acids in the protein, in order to better obtain amino acid embeddings.

In the perceptual representation learning method for protein conformations based on a pre-trained language model provided by the above embodiment, unlike an existing single-task pre-training model, a multi-task pre-training model using different pre-training tasks under different conformation symbols is proposed, allowing the model to calculate a sum of a plurality of losses, so as to learn more discriminative conformation symbol representations.

The specific implementation mentioned above provides a detailed description of the technical solution and beneficial effects of the present invention. It should be understood that the above is only the optimal embodiment of the present invention and is not intended to limit the present invention. Any modifications, supplements, and equivalent substitutions made within the scope of principle of the present invention should be included in the scope of protection of the present invention.

1. A perceptual representation learning method for protein conformations based on a pre-trained language model, comprising the following steps:
obtaining a protein made up of an amino acid sequence, building different data sets according to protein conformations, and defining a prompt for each type of protein conformation;
building, based on a pre-trained language model, a representation learning module for fusing an embedding representation of each type of the prompt into an embedding representation of the protein, so as to obtain a protein embedding representation under a prompt identifier;
building a task module for performing task prediction on a task corresponding to each type of protein conformation based on the protein embedding representation under the prompt identifier;
building a loss function for each type of task based on a task prediction result and a tag, and updating model parameters of the representation learning module and the task module in combination with loss functions of all types of tasks and the different data sets; and
after the model parameters are updated, extracting the representation learning module as a protein representation module.

2. The perceptual representation learning method for protein conformations based on a pre-trained language model according to claim 1, wherein the representation learning module comprises a prompt embedding layer, an amino acid embedding layer, a fusion layer, and the pre-trained language model, wherein the prompt embedding layer is configured to learn the embedding representation of each type of prompt, the amino acid embedding layer is configured to learn the embedding representation of the protein, the fusion layer is configured to fuse the embedding representation of each type of the prompt and the embedding representation of protein to obtain a fused representation, and the pre-trained language model is configured to perform representation learning on the fused representation to obtain the protein embedding representation under each type of the prompt identifier; and
the task module comprises a task mapping layer corresponding to each type of protein conformation, and each task mapping layer is configured to perform task prediction based on the protein embedding representation under each type of the prompt identifier.

3. The perceptual representation learning method for protein conformations based on a pre-trained language model according to claim 2, wherein the amino acid embedding layer comprises an amino acid information embedding layer and an amino acid position embedding layer which are configured to extract an amino acid information representation and an amino acid position representation according to the amino acid sequence, respectively, and the amino acid information representation and the amino acid position representation are superimposed to obtain the embedding representation of the protein.

4. The perceptual representation learning method for protein conformations based on a pre-trained language model according to claim 2, wherein the pre-trained language model is a pluggable masked pre-trained language model, wherein the masked pre-trained language model comprises BERT, RoBERTa, ALBERT, and XLNet.

5. The perceptual representation learning method for protein conformations based on a pre-trained language model according to claim 2, wherein the task mapping layer comprises a dual-layer MLP for performing task prediction based on the protein embedding representation under each type of the prompt identifier.

6. The perceptual representation learning method for protein conformations based on a pre-trained language model according to claim 1, wherein the loss function for each type of prediction task is to minimize an error of the task prediction result and the tag.

7. The perceptual representation learning method for protein conformations based on a pre-trained language model according to claim 1, wherein the protein representation module is applied to a prediction task for a protein structure and/or a protein function;
during application, in the protein representation module, embedding representations of all types of the prompts are simultaneously fused into the embedding representation of the protein, so as to obtain protein embedding representations under all the prompt identifiers; and the protein embedding representations under all the prompt identifiers are configured to predict the protein structure and/or the protein function.

8. The perceptual representation learning method for protein conformations based on a pre-trained language model according to claim 7, wherein the embedding representations of all types of the prompts being simultaneously fused into the embedding representation of the protein comprises:
splicing the embedding representations of all the types of the prompts first, and then performing fusion on an obtained spliced representation and the embedding representation of the protein, wherein the fusion comprises splicing, full connection mapping, and convolutional mapping.

9. The perceptual representation learning method for protein conformations based on a pre-trained language model according to claim 1, wherein the protein conformation comprises a natural folding state and an interaction state;
for the natural folding state, an amino acid sequence with a mask is taken as a sample and a normal amino acid sequence is taken as a sample tag to constitute a data set, a corresponding prompt in the natural folding state is a sequence prompt, and a corresponding task is a prediction task for a masked amino acid; and
for the interaction state, at least two amino acid sequences are taken as samples and a protein reaction type is taken as a sample tag to constitute a data set, a corresponding prompt in the interaction state is an interaction prompt, and a corresponding task is a prediction task for a protein interaction.
